(19) United States
(12) Patent Application Publication
(10) Pub. No.: US 2017/0367680 A1
(43) Pub. Date: Dec. 28, 2017

(54) ULTRASONIC SENSOR FOR HEALTH MONITORING

(71) Applicant: MiiCs & Partners (Shenzhen) Co., Ltd., Shenzhen (CN)

(72) Inventor: JUAN WANG, Shenzhen (CN)

(21) Appl. No.: 15/336,938

(22) Filed: Oct. 28, 2016

(30) Foreign Application Priority Data
Jun. 27, 2016 (CN) 201610477103.6

(51) Int. Cl. A61B 8/00; A61B 8/04; A61B 8/02; A61B 8/06; H01L 41/113; H01L 27/20

(52) U.S. Cl. CPC A61B 8/4281; H01L 41/1132; H01L 27/20; A61B 8/02; A61B 8/06; A61B 8/04; A61B 8/4236

(57) ABSTRACT

An ultrasonic sensor for monitoring a user's health characteristics, is flexible and close-fitting on the user's skin. The ultrasonic sensor includes a substrate, a signal transmitting layer positioned on the substrate, a signal receiving layer positioned on the substrate, and a flexible layer positioned on the signal receiving layer. The flexible layer is configured to attach to the user's skin. The signal transmitting layer includes a second electrode layer and a plurality of piezoelectric units formed on the second electrode layer. Each piezoelectric unit includes a second piezoelectric material layer formed on the second electrode layer and a conductive layer formed on the second piezoelectric material layer.

### FIELD

The subject matter herein generally relates to an ultrasonic sensor, particularly to an ultrasonic sensor for health monitoring.

### BACKGROUND

Ultrasonic sensors have many advantages such as small size, cheap price, safety, and widespread use in medical devices. The ultrasonic sensor for medical diagnosis is attached to a user's skin to emit ultrasonic waves. However, results obtained from the ultrasonic sensor may not be accurate when air is positioned between the ultrasonic sensor and the user's skin. Therefore, there is room for improvement in the art.

### BRIEF DESCRIPTION OF THE DRAWINGS

Implementations of the present technology will now be described, by way of example only, with reference to the attached figures.

FIG. 1 is cross-sectional view of a first exemplary embodiment of an ultrasonic sensor.

FIG. 2 is cross-sectional view of a signal transmitting layer of the ultrasonic sensor of FIG. 1.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, methods, procedures, and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

The term “coupled” is defined as connected, whether directly or indirectly through intervening components, and is not necessarily limited to physical connections. The connection can be such that the objects are permanently connected or releasably connected. The term “comprising” when utilized, means “including, but not necessarily limited to”; it specifically indicates open-ended inclusion or membership in the so-described combination, group, series, and the like.

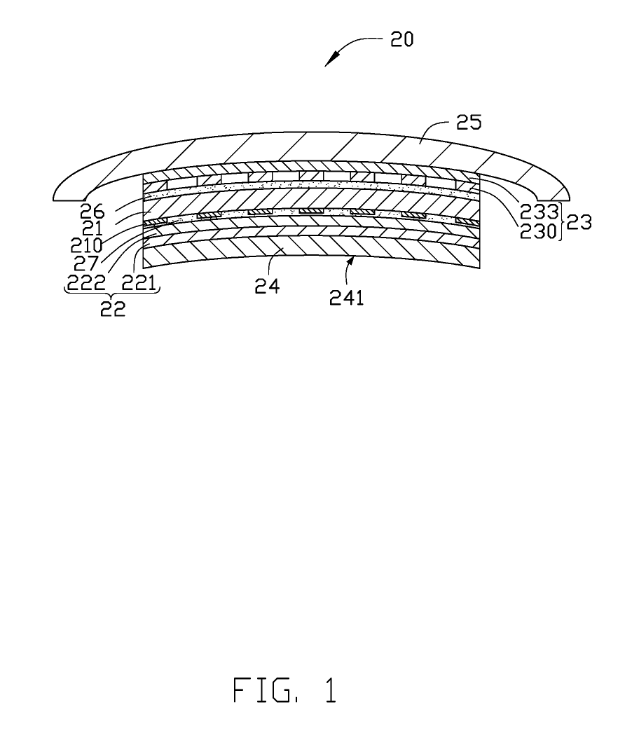

FIG. 1 illustrates an ultrasonic sensor 20 according to an exemplary embodiment. The ultrasonic sensor 20 can be used in an electronic device configured for placement on a user's skin and to assist in medical monitoring. A user's health characteristics such as blood flow, blood pressure, and heart rate can thus be constantly monitored.

The ultrasonic sensor 20 includes a substrate 21, a signal receiving layer 22, a signal transmitting layer 23, a flexible layer 24, and a protecting layer 154. In this exemplary embodiment, the ultrasonic sensor 20 has a curved shape (e.g. arc shape) to fit a user's body or a part of the body. The signal receiving layer 22 is coupled to a surface of the substrate 21 by a first adhesive layer 27. The signal transmitting layer 23 is coupled by a second adhesive layer 26 to a surface of the substrate 21 facing away from the signal receiving layer 22. In this exemplary embodiment, the first adhesive layer 27 and the second adhesive layer 26 are flexible.

The flexible layer 24 is formed on a surface of the signal receiving layer 22 facing away from the signal transmitting layer 23. The flexible layer 22 includes a contact surface 241 which faces away from the signal receiving layer 22. The contact surface 241 directly contacts the user's skin when using the ultrasonic sensor 20. The flexible layer 24 is configured to protect the signal receiving layer 22. The flexible layer 22 can be made of a common flexible material (e.g. latex or rubber). The flexible layer 22 can thus fit any part of the user's body and be close-fitting on the user's skin. In this exemplary embodiment, the flexible layer 22 has a curved shape (e.g. arc shape). In addition, the flexible layer 22 is soft to the touch, and comfortable to wear on the user's skin.

The protecting layer 25 is formed on a surface of the signal transmitting layer 23 facing away from the signal receiving layer 22. The protecting layer 25 is configured to protect the signal transmitting layer 23.

A plurality of thin film transistors 210 are formed on the substrate 150. The plurality of thin film transistors 210 are arranged in an array and are electrically coupled to the signal receiving layer 22. The thin film transistors 210 are configured to receive electrical signals from the signal receiving layer 22, convert the electrical signals to data signals for images or information in other form.

In this exemplary embodiment, the substrate 21 can be made of a flexible material, such as polyimide or polyethylene terephthalate. The substrate 21 has a curved shape (e.g. arc shape) to make the ultrasonic sensor 20 fit the user's body (e.g. arm or wrist). In some embodiments, the substrate 21 is made of a rigid material, such as glass, and has a curved shape (e.g. arc shape) to fit the user's body (e.g. arm or wrist).

The signal transmitting layer 23 is configured to emit ultrasonic waves continuously. The signal receiving layer 22 is configured to receive ultrasonic waves reflected by a human body or part to which the ultrasonic sensor 20 is attached. The signal receiving layer 22 includes a first piezoelectric material layer 221 and a first electrode layer 222 positioned on the first piezoelectric material layer 221. The first piezoelectric material layer 221 is coupled to the substrate 21 by the first adhesive layer 27. That is, the first adhesive layer 27 is positioned between the substrate 21 and the first piezoelectric material layer 221.

The signal transmitting layer 23 includes a second electrode layer 233 and a plurality of piezoelectric units 230 formed on the second electrode layer 233, wherein the piezoelectric units 230 are closer to the substrate 21 than the second electrode layer 233 to the substrate 21. The piezoelectric units 230 are separated from each other. Each piezoelectric unit 230 includes a second piezoelectric material layer 232 formed on the second electrode layer 233 and a conductive layer 231 formed on the second piezoelectric material layer 232 facing away from the second electrode layer 233. A method for making the piezoelectric units 230 may include the following steps: forming a continuous piezoelectric material layer (not shown) on the second electrode layer 233, forming a continuous conductive material layer (not shown) on the continuous piezoelectric material layer, and etching and patterning the continuous piezoelectric material layer and the continuous conductive material layer.

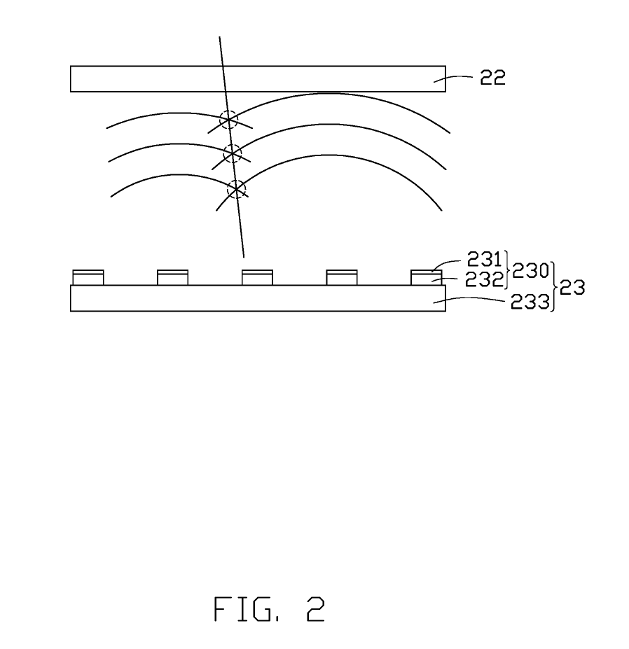

Referring now to FIG. 1 and FIG. 2, each second piezoelectric material layer 230 is able to vibrate and emit ultrasonic waves when a voltage is applied between the second electrode layer 233 and the corresponding conductive layer 231. Each piezoelectric unit 230 can emit ultrasonic waves independently, which is called “beam forming mode”. In the beam forming mode, ultrasonic waves emitted from one piezoelectric unit 230 overlap with ultrasonic waves emitted from other piezoelectric units 230, which effectively improve the intensity of the ultrasonic waves from the signal transmitting layer 23. In this embodiment, the piezoelectric units 230 can emit ultrasonic waves at a same time. In other embodiments, the piezoelectric units 230 can emit ultrasonic waves in a certain order, for example, the piezoelectric units 230 can emit ultrasonic waves in order from leftmost to rightmost. The piezoelectric units 230 can emit ultrasonic waves having a same intensity or different intensities.

The first piezoelectric material layer 221 and the second piezoelectric material layer 232 can be made of polyvinylidene fluoride (PVDF). The first electrode layer 222, the second electrode layer 233, and the conductive layer 231 can be made of a same electrically conductive material or different electrically conductive materials.

As an example of when in use, the ultrasonic sensor 20 is attached to a user's skin by attaching the flexible layer 24 on the user's skin (e.g. wrist). A voltage is applied between the second electrode layer 233 and the conductive layer 231, and the second piezoelectric material layer 232 vibrates and emits ultrasonic waves. The ultrasonic waves pass through the substrate 21, the signal receiving layer 22, the flexible layer 24, and the user's skin to reach internal organs or elements of the body, and a portion of the ultrasonic waves is reflected to the signal receiving layer 22. The reflected ultrasonic waves would be changed according to the status of the internal organs or elements of the body they encounter, thus vibration of the first electrode layer 222 would be changed. The signal receiving layer 152 converts the received ultrasonic wave signals to electrical signals and transmits the electrical signals to the thin film transistors 210. The thin film transistors 210 convert the electrical signals to data signals.

In other embodiments, the ultrasonic sensor 20 may further include a storage device (not shown). The results of monitoring (e.g. data signals) can be stored in the storage device. In some embodiments, the ultrasonic sensor 20 may be coupled to an outside readout device (not shown) by wires, WIFI, or BLUETOOTH, and the outside readout device may display the results as images or as information in other form. The outside readout device can be a mobile phone or a computer.

In some embodiments, the ultrasonic sensor 20 may be integrated with a display panel (not shown) and configured to display the data signals from the ultrasonic sensor 20. The user can observe the images or other information on the display panel. The display panel may be a known organic light emitting diode (OLED) display panel or a known liquid crystal display (LCD) panel. For example, the display panel may be a flexible OLED display panel, so the ultrasonic sensor 20 integrated with the display panel can be attached to the body or part of the user. The display panel may be an LCD panel having a curved shape to fit the body of the user.

When the ultrasonic sensor 20 is integrated with a display panel (not shown), a shielding layer (not shown) may be positioned between the ultrasonic sensor 20 and the display panel. The shielding layer may cover at least one surface of the ultrasonic sensor. The shielding layer may be made of an electrically conductive material and configured to shield the ultrasonic sensor 20 from electrical activity and radiation in the display panel. In addition, the shielding layer may be flexible. In some embodiments, the shielding layer 13 may cover at least two surfaces of the ultrasonic sensor 20, substantially enclosing the ultrasonic sensor 20 with a shield.

It is to be understood, even though information and advantages of the present embodiments have been set forth in the foregoing description, together with details of the structures and functions of the present embodiments, the disclosure is illustrative only; changes may be made in detail, especially in matters of shape, size, and arrangement of parts within the principles of the present embodiments to the full extent indicated by the plain meaning of the terms in which the appended claims are expressed.

What is claimed is:

1. An ultrasonic sensor for monitoring a user's health status, comprising:
a substrate;
a signal transmitting layer positioned on the substrate and configured to emit ultrasonic waves;
a signal receiving layer positioned on the substrate and configured to receive ultrasonic waves; and
a flexible layer positioned on signal receiving layer, the flexible layer configured to attach to the user's skin;
wherein the signal transmitting layer comprises a second electrode layer and a plurality of piezoelectric units spaced apart from each other and formed on the second electrode layer; and
wherein each piezoelectric unit comprises a second piezoelectric material layer formed on the second electrode layer, and a conductive layer formed on the second piezoelectric material layer.

2. The ultrasonic sensor of claim 1, wherein the signal receiving layer is positioned on a surface of the substrate; the signal transmitting layer is positioned on a surface of the substrate facing away from the signal receiving layer.

3. The ultrasonic sensor of claim 2, wherein the signal receiving layer is coupled to the substrate by a first adhesive layer; the signal transmitting layer is coupled to the substrate by a second adhesive layer.

4. The ultrasonic sensor of claim 3, wherein the first adhesive layer and the second adhesive layer are flexible.

5. The ultrasonic sensor of claim 1, wherein the signal receiving layer comprises a first piezoelectric material layer and a first electrode layer positioned on the first piezoelectric material layer.

6. The ultrasonic sensor of claim 5, wherein a plurality of thin film transistors is arranged in an array and formed on the substrate, the plurality of thin film transistors is electrically coupled to the signal receiving layer and is configured to receive electrical signals from the signal receiving layer.

7. The ultrasonic sensor of claim 1, wherein the ultrasonic sensor has a curved shape to fit the user's body.

8. The ultrasonic sensor of claim 1, wherein the flexible layer is made of a flexible material to flexibly and tightly attach to the user's skin.

9. The ultrasonic sensor of claim 1, further comprising a protecting layer on a surface of the signal transmitting layer facing away from the signal receiving layer.

10. The ultrasonic sensor of claim 1, wherein the substrate is made of a flexible material.

11. The ultrasonic sensor of claim 1, wherein the substrate is made of a rigid material and has a curved shape to fit the user's body.

12. An ultrasonic sensor for monitoring a user's health status, comprising:
a substrate;
a signal transmitting layer positioned on the substrate and configured to emit ultrasonic waves;
a signal receiving layer positioned on the substrate and configured to receive ultrasonic waves; and
a flexible layer positioned on signal receiving layer, the flexible layer being configured to attaching to the user's skin;
wherein the signal transmitting layer comprises a second electrode layer and a plurality of piezoelectric units formed on the second electrode layer, the plurality of piezoelectric units spaced apart from each other; and
wherein each piezoelectric unit is able to emit ultrasonic waves independently; ultrasonic waves emitted from one piezoelectric unit overlap with ultrasonic waves emitted from other piezoelectric units around.

13. The ultrasonic sensor of claim 12, wherein each piezoelectric unit comprises a second piezoelectric material layer formed on the second electrode layer and a conductive layer formed on the second piezoelectric material layer.

14. The ultrasonic sensor of claim 12, wherein the signal receiving layer is positioned on a surface of the substrate; the signal transmitting layer is positioned on a surface of the substrate facing away from the signal receiving layer.

15. The ultrasonic sensor of claim 12, wherein the signal receiving layer comprises a first piezoelectric material layer and a first electrode layer positioned on the first piezoelectric material layer.

16. The ultrasonic sensor of claim 12, wherein a plurality of thin film transistors is arranged in an array and formed on the substrate, the plurality of thin film transistors is electrically coupled to the signal receiving layer and is configured to receive electrical signals from the signal receiving layer.
